(19) United States
(12) Patent Application Publication
(10) Pub. No.: US 2012/0004431 A1
(43) Pub. Date: Jan. 5, 2012

(54) 2-Alkyl alkoxylated alcohol sulfonate

(75) Inventors: Paul D. Berger, Sugar Land, TX (US); Christie H. Berger, Sugar Land, TX (US)

(73) Assignee: OIL CHEM TECHNOLOGIES, Sugar Land, TX (US)

(21) Appl. No.: 13/199,809

(22) Filed: Sep. 10, 2011

Related U.S. Application Data
(62) Division of application No. 12/932,628, filed on Mar. 2, 2011.
(60) Provisional application No. 61/339,767, filed on Mar. 9, 2010.

(51) Int. Cl. C07D 301/00

(52) U.S. Cl. 549/513

(57) ABSTRACT

2-alkyl alkoxylated alcohol sulfonate surfactants having the structure R—O—(PO)x(EO)yCHR'CHR′CH₂SO₃Na are disclosed along with a process of making such surfactants. The surfactants are both electrolyte tolerant and tolerant to high temperatures and may be use for many applications where these properties are required.

### CROSS REFERENCE TO RELATED APPLICATIONS

This Application is based on provisional application Ser. No. 61/339,767, filed Mar. 9, 2010 and is a Divisional of Application 61/339,767.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

The present invention describes a composition and a process for the manufacture of electrolyte tolerant, high temperature tolerant 2-alkyl alkoxylated alcohol sulfonate surfactants.

U.S. Pat. No. 6,878,682 awarded to O'Lenick Jr. discloses a series of low foaming capped nonionic surfactants made by the reaction of methallyl chloride with the terminal hydroxyl of an alkoxylated alcohol. This reaction gives a series of nonionic surfactants of the following structure;

R—O—(CH₂CH2O)x—(CH₂CH(CH₃)O)y—CH₂—C(CH₃)═CH₂

Where

R is alkyl having 10 to 20 carbons

x is an integer ranging from 3 to 20,

Y is an integer ranging from 0 to 20.

This invention provides a means of introducing a terminal double bond to nonionics.

U.S. Pat. No. 4,275,013 awarded to Tokosh et al. discloses a process for the preparation of salts of alkane sulfonic acids by addition of alpha olefins to bisulfite in an organic cosolvent system consisting of water and an organic hydroxyl-containing compound in the presence of a free radical initiator. This invention discloses a process of sulfonating a terminal double bond.

Bright et al in Journal of Applied Chemical Biotechnology, Vol. 25, pages 901-912 (1975) further discusses the sulfonation of terminal double bonds of alkenes with sodium bisulfite.

Wu et al. in Tenside Surfactants Detergents Vol 47 page 3 (2010) discusses the advantages of branched alcohol propoxylated sulfate surfactants for Improved oil recovery.

Shupe in U.S. Pat. No. 4,886,120 discloses a method of removing residual oil from the near well-bore area of a subterranean formation using seawater containing a surfactant having the formula:

RO(C₃H₆O)₇(C₂H₄O)₂YX

Where

R is a mixture of alkyl groups containing from 12 to 15 carbon atoms,

Y is sulfate group, and

X is a monovalent cation.

The previous two patents use ether sulfates for Enhanced Oil Recovery (EOR) but these are not high temperature tolerant because the sulfates, being esters, are thermally unstable and subject to hydrolysis at high temperatures as is well known in the literature.

U.S. Pat. No. 4,856,589 awarded to Kuhlman et al. discloses a composition with the structure

RO—(R¹O)x(R²O)y—CH₂CH(R³)(R⁴)nSO₃⁻M⁺

Wherein:

R=a 6 to 16 carbon linear or branched alkyl radical or blend of alkyl radicals,

R¹=ethyl or i-propyl group,

R²=ethyl or i-propyl group,

R³=OH when R⁴=CH₂ or H when R⁴ is absent,

R⁴=CH₂ when R³=OH, or absent when R³=H,

M=sodium, potassium, or ammonium,

x=0-9

y=0-9

n=0-1.

Wellington et al. in U.S. Pat. No. 4,502,538 disclose a polyoxy aliphatic sulfonate surfactant with the formula

RO(R′O)xCH₂CHOHCH₂SO₃M,

made by reacting an alkoxylated alcohol with epichlorohydrin in the presence of stannous chloride to yield and alkoxy glycerol chloride. The glycerol chloride is converted to an epoxide and then sulfonate with aqueous sodium hydroxide and sodium bisulfite. R is a C8 to C18 alyl radical, R′ is an ethylene radical, and x is 3 to 18 or R is a C12 to C15 alkyl radical, R′ is an ethylene radical, x is 7 to 25 and M is a sodium cation. As noted in the Wellington patent U.S. Pat. No. 4,502,538, these alcohol ethoxylated glycerol sulfonates are available from Shell Chemical and sold under the tradename NEGS for NEODOL Ethoxylate Glyceryl Sulfonates.

Although the last two references use ether sulfonates for EOR, these sulfonates differ in structure from the present invention and the method of synthesizing them results in undesireable by-products.

s Cummins in Chapter 8 of Handbook of Detergents Part F: Production, on page 160, discusses the polysulfonate byproducts formed when producing glycerol ether sulfonates using epichlorohydrin. This is also discussed in U.S. Pat. No. 6,133,474 awared to Rasheed et al.

The present invitation discloses new 2-alkyl alkoxylated alcohol sulfonates and a process for making same. These surfactants provide excellent high tempereature and high salinity tolerance while not being subject to some of the problems such as poor yields, by-products and high costs associated with the prior art. These novel surfactants can be applied to many applications including, but not limited to, enhanced oil recovery (EOR), agricultural emulsions, metalworking, textiles, mining, paper and pulp processing, and other applications where high temperature, high electrolyte tolerant surfactanats are required.

### BRIEF DESCRIPTION OF THE INVENTION

This invention discloses an electrolyte tolerant and high temperature tolerant family of surfactants and a process to produce such surfactants.

The invention involves the reaction of an alkoxylated alcohol or alkylphenol, first with methallyl chloride or a similar alkylsubstituted allyl chloride to form a terminally unsaturated intermediate. This intermediate is then sulfonated with a bisulfite or metabisulfite salt to form the corresponding alkyl or alkylphenol ether sulfonate.

One of the objects of the present invent is to provide a group of surfactants that are tolerant to both high temperature and high electrolyte concentration.

Another object of the present invention is to provide a process to make ether sulfonates eliminating the byproducts associated with previous processes disclosed in the literature.

Other advantages of the present invention will become apparent to those skilled in the art with the benefit of the following detailed description of embodiment.

### DETAILED DESCRIPTION OF THE PREFERRED EMBODIMENTS

The process for making the family of surfactants of the present invention starts with an alcohol or phenolic compound that is alkoxylated with propylene oxide followed by ethylene oxide or with ethylene oxide alone as shown in structure (1) below where PO is methyl oxirane and EO is oxirane.

R—O—(PO)x(EO)y (1)

and where R is a linear or branched alkyl or alkenyl group containing from 6 to 60 carbons or R is an substituted phenol group where the substitute on the phenol is linear or branched alkyl or alkenyl containing from 4 to 30 carbons,

x=0 to 100, and

y=0 to 30.

The amounts of PO and EO are chosen to give the desired electrolyte tolerance required for a particular application. Both the amount of PO and EO are also chosen to give the desired performance including but not limited to interfacial tension lowering

(IFT), wetting, detergency, dispersion, and emulsification.

Examples of phenolic compounds include, but are not restricted to, alkylphenols, dialkylphenols. Non-exclusive examples of alcohols that may be used include, but are not restricted to, linear or branched petroleum based or natural alcohols such as those sold under the Neodol trademark by Shell Chemical or the Alfol trademark by Sasol.

In structure 1, when x is greater than 0, this invention requires that y be an integer high enough that a majority of the propylene oxide end groups (>95%) be covered by at least one EO group. This is necessary in order to proceed with the sulfonation step.

Sulfonation is accomplished by first reacting the compound described by structure (1) with KOH and methallyl chloride as described by O'Lenick, Jr. in U.S. Pat. No. 6,878,682. This forms an intermediate product with a terminal unsaturation such as shown in (2) below.

R—O—(PO)x(EO)y+KOH+CH₂═C(CH₃)CH₂Cl→R—O—(PO)x(EO)yCH₂C(CH₃)═CH₂+KCl (2)

Compound (2) is subsequently reacted with sodium bisulfite in the presence of a free radical catalyst to form the final ether sulfonate (3). Other allyl chloride derivatives can be used, including but not restricted to ethylallyl chhlride, propylallyl chloride, butylallyl chloride, but these are generally not preferred because of limited commercial availability and cost.

R—O—(PO)x(EO)yCH₂C(CH₃)═CH₂+NaHSO_{3→R—O—(PO)x(EO)yCH(CH}₃)CH₂SO₃Na (3)

The reaction using methallyl chloride only proceeds giving high yields when all or a majority of primary hydroxyls are present and therefore only the intermediates of structure (1) having at least one terminal EO will react. Generally, we have found that at least 5 EO units are required to insure that enough terminal secondary propoxyl groups have been covered.

Sodium metabisulfite may be used along with sodium hydroxide in place of sodium bisulfite. The combination of sodium metabisulfite and sodium hydroxide in water will form sodium bisulfate by the following reaction where the hydroxide acts as a catalyst.

Na₂S₂O₅+NaOH+H₂O→2NaHSO₃+NaOH

The inventors of the present invention have found that compounds described by structure (3) are very effective in lowering interfacial tensions between oil and aqueous solutions such as seawater and brines. This makes them extremely useful as surfactants for Enhanced Oil Recovery (EOR). In addition the presence of the sulfonate group renders the structure tolerant to high temperatures in contrast to similar ether sulfates that have been employed and described in the literature.

If allyl chloride is used in place of methallyl chloride the intermediate has the structure

R—O—(PO)x(EO)yCH₂CH═CH₂. (4)

and the final sulfonate has the structure shown as (5) below where R, x, y and M have the same meaning and have been defined previously.

R—O—(PO)x(EO)yCH₂CH₂CH₂SO₃M. (5)

However, this reaction also forms 1-sodium sulfonate-2-sulfinic acid as shown in structure (6) below and 1,2 disodium sulfonates as shown in structure (7) below and as described by Herke and Rasheed in JAOCS, Vol. 69, no. 1 pg 47-51 (1992) and Bright et al. in Journal of Applied Chemical Biotechnology, 1975, 25, 901-912. By replacing the hydrogen on the second carbon with a methyl group we have found the reactions producing byproducts are blocked.

R—O—(PO)x(EO)yCH₂CH(SO₂H)CH₂SO₃M (6)

R—O—(PO)x(EO)yCH₂CH(SO₃M)CH₂SO₃M (7)

The invention includes a composition and a process to make such a composition where the structure of the composition is

R—O—(PO)x(EO)yCHR'CH₂SO₃M

Where R=branched or linear alkyl or alkenyl having 6 to 60 carbons or R is a substituted phenolic compound having one or more linear or branched alkyl or alkenyl substitutions of 4 to 30 carbons in length,

PO=methyl oxirane,

EO=oxirane,

x=0-100,

y=0-30,

R′=CH₃, and,

M=a monovalent cation.

In addition to the structure of the surfactant of the present invention being different from those reported in the literature, the process for making this surfactant involves the use of less toxic methallyl chloride.

The following examples further illustrate the object and desirable properties obtained with the present invention.

### EXAMPLE 1

### Preparation of a 2-methyl Alkoylated Alcohol Sulfonate Containing Propylene and Ethylene Oxide

0.200 Mole (296 grams) of a C12-14 linear alcohol alkoxylate having 16 moles of propylene oxide and 8 moles of ethylene oxide (C12-14+16PO+8EO) supplied by Huntsman Chemical Company was added to a 4 necked 1000 ml round bottom flask fitted with a condenser, temperature controller, stirrer and Nitrogen dip tube. A stream of Nitrogen was bubbled through the alcohol alkoxylate. While stirring and maintaining the Nitrogen bubble, 0.200 moles of potassium hydroxide pellets (11.20 g) was added and the mixture heated to 90° C. After all the potassium hydroxide was reacted as evidence by the disappearance of any pellets, the solution was cooled to below 70° C. and 0.200 Moles of methallyl chloride (18.12 g) was added and the mixture heated and allowed to continue to react at 90′° C. for 8 hours. After 8 hours the mixture was cooled and the potassium chloride formed during the reaction was removed by centrifuging and the clear light yellow intermediate product was used for further reaction. 0.150 Moles of the clear intermediate product (228.1 g) was added back to the flask. In a separate container 0.500 Moles of sodium meta-bisulfite (95.0 g) along with 0.250 moles (10.0 g) of sodium hydroxide was added to 600 grams of water and mixed until the sodium meta-bisulfite and the sodium hydroxide had dissolved. This solution was then added to the flask containing the intermediate and heated to 80° C. under a Nitrogen purge. 0.125 Moles (24.3 g) of tert-Butyl peroxybenzoate was carefully added and the mixture allowed to exotherm until the temperature no longer rose. The mixture was then heated to 90° C. and allowed to continue to react. Samples were taken periodically beginning after 3 hours reaction time to determine the extent of completeness of the reaction by measuring the anionic activity using the 2-phase titration method with Hyamine 622 known to those familiar with the art. The reaction was complete when the activity equaled the theoretical value (25.4% assuming 1636 to be the Equivalent Weight of the product)) or when no change in activity was detected in two subsequent samples taken 1 hour apart.

Table 1 below shows the progress of the reaction.

TABLE 1
Progress of reaction

| Reaction Time, | Anionic Active, | Activity, | Conversion, |
|---|---|---|---|
| hr | me/g | wt % | % |
| 3 | 0.107 | 17.4 | 68.5 |
| 4 | 0.150 | 24.3 | 95.7 |
| 5 | 0.153 | 24.8 | 97.6 |
| 6 | 0.153 | 24.8 | 97.6 |

### EXAMPLE 2

### Preparation of a 2-methyl Ethoxylated Alcohol Sulfonated Surfactant

567.0 g (1.00 Mole) Neodo™ 67-7, a C16-17 alcohol reacted with 7 moles of ethylene oxide, a product of Shell Chemical were combined with 56.1 g (1.00 Mole) potassium hydroxide and heated with stirring and a Nitrogen purge until all the potassium hydroxide had reacted. The solution was then cooled to below 70° C. and 90.6 (1.00 Mole) methallyll chloride was added. The mixture was then carefully heated and held at 90° C. for 8 hours. The material was then allowed to cool to room temperature and centrifuged to remove the potassium chloride formed. 413.g (0.664 Moles) of the clear liquid after centrifugation was added to a flask. In a separate container 8.86 g (0.221) sodium hydroxide and 84.0 g (0.442 Moles) of sodium meta-bisulfite were dissolved in 466 g of water and added to the flask. Finally 28.0 grams (0.144 Moles) tert-butyl peroxybenzoate was added and the entire mixture heated and held at 80° C. and allowed to exotherm. Once the exotherm had ceased, the mixture was heated to 90° C. and held at temperature while monitoring the anionic activity. After 2 hours the activity was 17.4 wt %. After 4 hours it was 24.3 wt %. After 6 hours the activity was 28 wt % and further reaction did not result in any increase.

### EXAMPLE 3

### Evaluation of the 2-methyl Alkoxylated Alcohol Sulfonate Surfactant from Example 1 as an EOR Surfactant

This example described the IFT results obtained for a crude oil and a solution of the composition of the present invention in seawater It is well known by those familiar with the art that a low interfacial tension reaching less than 0.02 mN/m is required to release oil from the microscopic capillaries in the reservoir rock where it is trapped.

A synthetic sea water sample was prepared according to the formulation shown in Table 2.

TABLE 2
Synthetic Seawater

| Ingredient | % by wt |
|---|---|
| NaCl | 3.00 |
| MgCl₂•6H²O | 3.00 |
| CaCl₂•2H₂O | 2.00 |
| Water | 92.00 |

A 0.1, 0.2 and 0.3 wt % solutions of the formulation from Example 1 was prepared in the synthetic seawater described in Table 2. The interfacial tension of the solutions against a Southeast Asian crude oil was measured at 90° C. and the results are shown in Table 3 below.

TABLE 3
IFT, mN/m, at various concentrations and times

| | 5 minutes | 15 minutes | 30 minutes | 60 minutes | 120 minutes |
|---|---|---|---|---|---|
| 0.05 | 0.022 | 0.011 | 0.012 | 0.013 | 0.013 |
| 0.10 | 0.008 | 0.005 | 0.0045 | 0.0042 | 0.0044 |
| 0.20 | 0.011 | 0.008 | 0.0066 | 0.0076 | 0.0081 |

### EXAMPLE 4

### High Temperature Solubilities

This example demonstrates the high temperature stability and solubility of surfactants of the invention. Samples of various 2-methyl alkoxylated alcohol sulfonates were prepared by the method described in Example 1 and their solubilities at 0.2% wt % in various electrolyte solutions at 90° C. were measured.

TABLE 4
Solubilities at elevated temperatures

| Ether Sulfate | 15% NaCl | 20% NaCl | Synthetic Seawater |
|---|---|---|---|
| C12-15 + 8EO | Soluble | Soluble | Soluble |
| C12-15 + 5PO + 8EO | Soluble | Soluble | Soluble |
| C12-15 + 10PO + 8EO | Soluble | Soluble | Soluble |
| C12-15 + 16O + 8EO | Soluble | Soluble | Soluble |
| C12-15 + 20PO + 8EO | Soluble | Dispersible | Soluble |

Further embodiments and alternative embodiments of various aspects of the present invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiment. Elements and materials may be substituted for those illustrated and described herein, parts and processes may be reversed, and certain features of the invention may be utilized independently, as would be apparent to those skilled in the art after having benefited by this description of the invention. Changes may be made in the elements described herein without departing from the spirit and scope of the invention as described in the flowing claims. In addition, it is to be understood that features described herein independently may, in certain embodiments, be combined.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed:

1. A process for making electrolyte and high temperature tolerant 2-alkyl alkoxylated alcohol sulfonate surfactants for use in Enhanced Oil Recovery having the structure
R—O—(PO)x(EO)yCHR′CH₂SO₃M,
Where,
R=branched or linear alkyl or alkenyl having 6 to 60 carbons or R is a substituted phenolic compound having one or more linear or branched alkyl substitutions of 4 to 30 carbons in length,
PO=methyl oxirane,
EO=oxirane,
x=0-100,
y=0-30,
R′=CH₃, and
M=a monovalent cation, by,
(a) reacting an alkoxylated alcohol or alkyoxylated alkyl phenol first with or methallyl chloride and potassium chloride to form a terminally unsaturated intermediate,
(b) further reacting the intermediate with a bisulfite or a sulfite salt of a monovalent cation.

2. The process for making electrolyte and high temperature tolerant ether sulfonates as described in claim 3 where the monovalent cation M is Na.

3. The process for making electrolyte and high temperature tolerant ether sulfonates described in claim 3 where y is an integer large enough to insure that a majority of the terminal PO groups have been covered when x is greater than zero.
